(19) United States
(12) Patent Application Publication
(10) Pub. No.: US 2019/0000925 A1
(43) Pub. Date: Jan. 3, 2019

(54) COMPOSITION FOR TREATING OR PREVENTING INFLAMMATORY DISEASE CONTAINING COMPLEX OF INTERLEUKIN-15/INTERLEUKIN-15 RECEPTOR ALPHA-FC

(71) Applicant: Ajou University Industry-Academic Cooperation Foundation, Gyeonggi-do (KR)

(72) Inventor: Seonghyang Sohn, Gyeonggi-do (KR)

(21) Appl. No.: 15/949,993

(22) Filed: Apr. 10, 2018

(30) Foreign Application Priority Data
Jun. 30, 2017 (KR) 10-2017-0083617

(51) Int. Cl. A61K 38/20; A61P 31/22; A61K 38/17; A61K 9/00

(52) U.S. Cl. CPC A61K 38/2086; A61K 9/0014; A61K 38/1793; A61P 31/22

(57) ABSTRACT

The present disclosure relates to a composition for treating or preventing an inflammatory disease, and more particularly, to a composition for treating or preventing a herpes simplex virus infection containing interleukin-15 and interleukin-15 receptor alpha-Fc.

### CROSS-REFERENCE TO RELATED APPLICATIONS

The priority under 35 USC § 119 of Korean Patent Application 10-2017-0083617 filed Jun. 30, 2017 is hereby claimed. The disclosure of Korean Patent Application 10-2017-0083617 is hereby incorporated herein by reference, in its entirety, for all purposes.

### TECHNICAL FIELD

The present invention relates to a composition for treating or preventing an inflammatory disease, and more particularly, to a composition for treating or preventing a herpes simplex virus infection containing interleukin-15 and interleukin-15 receptor alpha-Fc.

### BACKGROUND ART

A herpes simplex virus (HSV) is a herpesviridae virus, and refers to a herpes simplex virus type 1 (HSV-1) and a herpes simplex virus type 2 (HSV-2). The HSV-1 and HSV-2 infections are the most common virus infections in the world. The HSV-1 infection is often acquired in infancy as a subclinical infection, while a subset involves a severe disease. HSV-2 is generally acquired by sexual activity and causes lesions primarily in a genital area. Infection caused by herpes virus causes several different diseases based on the infected area. A typical symptom of herpes virus is oral herpes (herpes simplex labialis) called a mouth rash or fever blister and occur when face or mouth is infected. Genital herpes (genital herpes simplex) is the second most common form of herpes, and other diseases such as herpetic whitlow, herpes gladiatorum, ocular herpes (herpes simplex correa or herpes simplex keratitis), brain herpes infectious encephalitis, Mollaret's meningitis, neonatal herpes, etc., are all caused by herpes simplex virus.

Currently, it is general to use a virustatic agent in an antiviral HSV therapy. The most common virus inhibitors (e.g., acyclovir, penciclovir, foscarnet, and idoxuridine) are nucleoside or pyrophosphate analogs of which general activation principle is based on inhibition of DNA synthesis in viral infected cells. In a double-blind, placebo-controlled study of 1385 patients with acute herpes labial simplex infection, acyclovir (Zovirax Creme form) was shown to reduce infection up to 0.5 day (i.e., from 5 days to 4.5 days) when administered five times a day for 4 days compared to placebo-treated patients. Moreover, this treatment has disadvantage in that occurrence of typical lesions in herpes is not able to be prevented. Recently, murine and correspondingly humanized antibody that specifically recognize glycoprotein B (gB) of HSV type 1 (HSV-1) and of HSV-2 are specifically identified. HSV-gB is an indispensable part of a multicomponent fusion system required for viral invasion and cell-cell fusion. It has been proven that monoclonal antibody MAb2c neutralizes the virus by blocking diffusion from virus cells to cells and a major mechanism avoiding humoral immune surveillance independent from antibody-dependent cell cytotoxicity (ADCC) and/or complement-dependent cytotoxicity (CDC) by HSV-1/2 [Eis-Hubinger et al., Intervirology 32:351-360(1991); Eis-Hubinger et al., Journal of General Virology 74:379-385(1993); WO2011/038933 A2; Krawczyk A, et al., Journal of Virology (2011); 85(4):1793-1803; Krawczyk, A., et al., Proc. Natl. Acad. Sci. USA (2013); 110(17):6760-6765).

International Publication WO 2005/023303 discloses a method of treating HSV by intravenous administration of an IgA fraction of human plasma or an IgG fraction of human plasma, but describes topical administration, herpes labialis, and herpes genital disease. Even though topical administration of an antibody has been described previously, this application is merely proposed for prophylactic use in prevention of sexually transmitted HSV-2 disease. In addition, prophylactic topical passive immunoprotection of a female mouse against genital herpes has been reported in a mouse model of vagina-transmitted HSV-2 infection by a monoclonal antibody against HSV-2 (Sherwood et al., Nat Biotechnol. 14 (4): 468-471 (1996)). Similarly, prophylactic topical administration of anti-HSV-2 antibody has also been reported in prevention of sexual transmission of HSV-2 (Zeitlin et al., Virology 225(1):213-215, 1996; Zeitlin et al., Contraception 56(5):329-335, 1997, Zeitlin et al., J. Reprod. Immunol. 40(1):93-101, 1998; Zeitlin et al., Nat. Biotechnol. 16:1361-1364, 1998).

In addition, document [Clement et al., ARVO, Abstract/Poster 6155/D1015] describes local administration of antibody-targeted phosphatidylserine (PS) in a rabbit model of acute HSV-1 keratitis, and document [Yu et al., Eye Science 12(3):145-150(1996)] describes topical use of an anti-HSV-monoclonal glycoprotein antibody in an acute herpetic keratitis of a rabbit infected by HSV-1. International Publication No. WO 2010/128053 discloses use of an antibody fragment that binds to a virus surface antigenic glycoprotein D that neutralizes HSV-1 and HSV-2 for topical ocular administration to treat eye diseases such as ocular keratitis. However, these treatments have difficulty in performing fundamental treatment as a symptomatic treatment by topical administration.

Meanwhile, as a case where IL-15 or its receptor is used for therapeutic use, a case where an IL-15 heterodimer protein (IL-15/IL-15Rα) is used as a therapeutic agent for inflammatory disease (Korean Patent Publication No. 10-2016-0103058), and a case where a conjugate including interleukin 15 and a sushi domain of IL-15Rα is used for cancer treatment (Korean Patent Laid-open Publication No. 10-2014-0062030), a cancer treatment method using recombinant radiated cancer cells simultaneously expressing human IL-15/human IL-15Rα (U.S. Patent Application Publication No. 2011-0081311 A1) and a method of treating Behcet's disease by modulating a level of IL-15/IL-15Rα specific to serum and organs (Petrushkin, H. et al., Front. Immunol., 6, 1˜6, 2015) are disclosed. However, a technology of using interleukin-15 or its receptor to treat HSV viral infections has not yet been reported.

Therefore, the present inventors made intensive efforts to develop a therapeutic agent for treating a fundamental HSV viral infection which is capable of being administered intravenously as well as topical administration, and as a result, found that when interleukin-15 and interleukin-15 receptor alpha-Fc protein complex was intravenously administered to an HSV-infected mouse, symptoms caused by HSV infection are improved by inhibiting maturation of NK cells in spleen cells, and completed the present invention.

### Technical Problem

An object of the present invention is to provide a composition for treating or preventing a herpes simplex virus infection.

### Technical Solution

In order to achieve the foregoing objects, the present invention provides a composition for treating or preventing a herpes simplex virus infection containing interleukin-15 and interleukin-15 receptor alpha-Fc.

Further, in order to achieve the foregoing objects, the present invention provides a method for treating or preventing infectious disease caused by herpes simplex virus, the method comprises administering a composition containing interleukin-15 and interleukin-15 receptor alpha-Fc to a subject in need of treatment or prevention of said disease.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows expression of IL-15 and IL-15Rα in an HSV-infected mouse. Image A shows RT-PCR results and graph B shows flow cytometry results.

FIG. 2: panel A shows the RT-PCR confirmation result of expression of IL-15Rα mRNA in RAW 264.7 cells transfected with IL-15Rα expression vector (pIL-15Rα) and IL-15/IL-15Rα expression vector (pIL-15/15Rα); graph B shows the confirmation result of frequency of IL-15Rα+ cells in peritoneal macrophage in a group in which pIL-15/15Rα is administered in a healthy normal mouse; graph C shows a disease severity score change by injection of pIL-15/15Rα; and panel D is an image showing a change in symptom after pIL-15/15Rα is injected in a mouse.

FIG. 3 shows FACS confirmation results of frequency of IL-15Rα+ cells in spleen and peripheral blood mononuclear cells (PBMC) of a symptomatic mouse group in which recombinant protein IL-15/IL-15Rα-Fc complex is treated intravenously.

FIG. 4 shows FACS results of immune markers in the spleen of the symptomatic mouse by treatment with recombinant protein IL-15/IL-15Rα-Fc complex, particularly it shows frequency of CD122+ cells (IL-15R beta) and frequency of CD132+ cells (IL-15R gamma).

FIG. 5 shows frequency of CD25+/Foxp3+ cells in the spleen of the symptomatic mouse by treatment with the recombinant protein IL-15/IL-15Rα-Fc complex.

FIG. 6: graph A shows frequency of CD3-NK1.1+ cells in lymphocytes in the symptomatic mouse by treatment with the recombinant protein IL-15/IL-15Rα-Fc complex; graph B shows frequency of CD11b−/CD27− cell, CD11b−/CD27+ cell and CD11b+/CD27+, and CD11b+/CD27− cell in the symptomatic mouse by treatment with the recombinant protein IL-15/IL-15Rα-Fc complex; graph C shows frequency of CD3-NK1.1+ cells in whole leukocytes in the symptomatic mouse by treatment with the recombinant protein IL-15/IL-15Rα-Fc complex; and graph D shows frequency of CD11b−/CD27− cell, CD11b−/CD27+ cell and CD11b+/CD27+, and CD11b+/CD27− cell in the symptomatic mouse by treatment with the recombinant protein IL-15/IL-15Rα-Fc complex.

FIG. 7 shows the frequency of IL-15Rα+ cells in PBMC of the symptomatic mouse treated with Colchicine and Infliximab.

### BEST MODE

In the present invention, it was confirmed that an HSV symptomatic model mouse infected with HSV type 1 virus shows oral and genital ulcers, erythema, skin pustules, skin ulcers, arthritis, diarrhea, red eyes, eyesight deterioration, loss of balance, discoloration, and facial edema, etc., and an interleukin-15/interleukin-15 receptor alpha-Fc protein complex was intravenously administered to the symptomatic mouse group, and thus it was confirmed that infection symptoms were substantially alleviated, and it was confirmed through flow cytometry that in the spleen and peripheral blood mononuclear cells (PBMC) of the symptomatic mouse group treated with interleukin-15 and interleukin-15 receptor alpha-Fc protein, frequency of IL-15Rα+ cells was increased, and frequencies of CD122+ cells (IL-15R beta), CD132+ cells (IL-15R gamma) and CD25+/Foxp3+ cells were increased. In addition, frequencies of CD11b−/CD27− cells, CD11b−/CD27+ cells, and CD11b+/CD27+ cells were up-regulated by treatment of the recombinant protein IL-15/IL-15Rα-Fc complex in the symptomatic mouse, but it was confirmed that the frequency of CD11b+/CD27− cells were down-regulated in whole cells of PBMC and lymphocytes of PBMC. CD11b+/CD27− cell population is a group of terminally differentiated NK cells in four stages of NK cell differentiation, and it was confirmed that treatment of IL-15/IL-15Rα-Fc complex inhibited NK cell terminal maturation to rejuvenate the NK cells.

Accordingly, an aspect of the present invention provides a composition for treating or preventing infectious disease caused by a herpes simplex virus containing interleukin-15 and interleukin-15 receptor alpha-Fc.

In the present invention, the interleukin-15 may have a concentration of 0.01 to 10 mg/mL, and the interleukin-15 receptor alpha-Fc may have a concentration of 0.01 to 60 mg/mL.

In the present invention, a content ratio of the interleukin-15 and interleukin-15 receptor alpha may be 0.1 to 5:5 to 50.

In the present invention, the herpes simplex virus infection may be selected from the group consisting of herpes labialis, angular herpes, genital herpes simplex, ocular herpes, brain herpes infectious encephalitis, Mollaret's meningitis, and neonatal herpes.

A carrier used in a pharmaceutical composition of the present invention includes a pharmaceutically acceptable carrier, adjuvant and vehicle, and is collectively referred to as a “pharmaceutically acceptable carrier”. The pharmaceutically acceptable carrier that may be used in the pharmaceutical composition of the present invention includes, but is not limited to, ion exchange, alumina, aluminum stearate, lecithin, serum protein (e.g., human serum albumin, buffer materials (e.g., various phosphates, glycine, sorbic acid, potassium sorbate, a partial glyceride mixture of saturated vegetable fatty acid), water, salt or electrolyte (e.g., protamine sulfate, disodium hydrogen phosphate, potassium hydrogen phosphate, sodium chloride and zinc salt), colloidal silica, magnesium trisilicate, polyvinylpyrrolidone, a cellulose-based substrate, polyethylene glycol, sodium carboxymethylcellulose, polyarylate, wax, polyethylene-polyoxypropylene-barrier polymer, polyethylene glycol, and wool fat, etc.

A route of administration of the pharmaceutical composition according to the present invention may be, but is not limited to, oral, intravenous, intramuscular, intraarterial, intramedullary, intrathecal, intracardiac, transdermal, subcutaneous, intraperitoneal, intranasal, enteral, topical, sublingual or rectal administration.

Oral administration and parenteral administration are preferred. The term “parenteral” as used herein includes subcutaneous, intradermal, intravenous, intramuscular, intraarticular, intramyeloid, intrasternal, intrathecal, intralesional and intracranial injection or infusion techniques.

The pharmaceutical composition may be in the form of a sterile injectable preparation, either as a sterile injectable aqueous or oily suspension. The suspension may be formulated according to techniques known in the art using suitable dispersants or wetting agents (e.g., Tween 80) and suspending agents. The sterile injectable preparation may also be a sterile injectable solution or suspension in a non-toxic parenterally acceptable diluent or solvent (e.g., a solution in 1,3-butanediol). Examples of the vehicle and solvent that are acceptably usable include mannitol, water, a Ringer solution, and an isotonic sodium chloride solution. In addition, sterile and non-volatile oil is conventionally employed as a solvent or a suspending medium. For this purpose, any non-volatile oil with low irritation, including synthetic monoglyceride or diglyceride, may be used. Natural oils in which fatty acids such as oleic acid and its glyceride derivative are pharmaceutically acceptable (e.g., olive oil or castor oil), especially polyoxyethylated forms thereof, are also useful in injection formulations.

The pharmaceutical composition of the present invention may be orally administered in any dosage form that are orally acceptable, including, but not limited to, a capsule, a tablet, and an aqueous suspension, and a solution. In the oral tablet, lactose and corn starch are included as commonly used carriers. A lubricant such as magnesium stearate is also typically added. For oral administration in a capsule form, lactose and dried corn starch are included as useful diluents. When the aqueous suspension is administered orally, an active ingredient is combined with an emulsifying agent and the suspending agent. If desired, a sweetener and/or a flavor and/or a colorant may be added.

The pharmaceutical composition of the present invention may also be administered in the form of suppositories for rectal administration. The composition of the present invention may be prepared by mixing with a suitable non-irritant excipient which is a solid at room temperature but is liquid at a rectal temperature. The material includes, but is not limited to, cocoa butter, beeswax, and polyethylene glycol.

Oral administration of the pharmaceutical composition according to the present invention is particularly useful when the desired treatment is associated with a site or an organ that is accessible by topical application. When topically applied to the skin, the pharmaceutical composition maybe formulated into a suitable ointment containing the active ingredient suspended or dissolved in the carrier. A carrier for topical administration of the composition of the present invention includes, but is not limited to, mineral oil, liquid paraffin, white petrolatum, propylene glycol, polyoxyethylene, polyoxypropylene compound, emulsified wax and water. Alternatively, the pharmaceutical composition may be formulated into a suitable lotion or cream containing the active compound suspended or dissolved in a carrier. A suitable carrier includes, but is not limited to, mineral oil, sorbitan monostearate, polysorbate 60, cetyl ester wax, cetearyl alcohol, 2-octyldodecanol, benzyl alcohol, and water. The pharmaceutical compositions of the present invention may also be topically applied to a low intestinal tract by rectal suppositories or by a suitable enema. A topically applied transdermal patch is also included in the present invention.

The pharmaceutical composition of the present invention may be administered by intranasal aerosol or by inhalation. The composition may be prepared according to techniques well known in the art of pharmacy, and may be prepared as a solution in brine using benzyl alcohol or other suitable preservatives, absorption enhancers for enhancing bioavailability, fluorocarbons and/or other solubilizing agents or dispersants known in the art.

The composition of the present invention may be used in combination with a conventional anti-inflammatory agent or in combination with a matrix metalloproteinase inhibitor, a lipoxygenase inhibitor, and an inhibitor of cytokines other than IL-1beta. The composition of the present invention may also be administered in combination with an immunomodulator (e.g., bropirimine, anti-human alpha interferon antibody, IL-2, GM-CSF, methionine enkephalin, interferon alpha, diethyldithiocarbamate, tumor necrosis factor, naltrexone and rEPO) or prostaglandin in order to prevent or combat IL-1-mediated disease symptoms such as inflammation. When the composition of the present invention is administered in combination with other therapeutic agents, the composition may be administered to a patient sequentially or concurrently. Alternatively, the pharmaceutical composition according to the present invention may be prepared by mixing a composition of the present invention with another therapeutic agent or prophylactic agent as described above.

The pharmaceutical composition of the present invention may be used for treating an infectious disease, particularly, a disease associated with viral infection. A disease state that may be treated or prevented by the pharmaceutical composition of the present invention includes, but is not limited to, herpes labialis, angular herpes, genital herpes simplex, ocular herpes, brain herpes infectious encephalitis, Mollaret's meningitis, and neonatal herpes, tuberculosis, gonorrhea, typhoid, meningitis, osteomyelitis, meningococcal infection, endometritis, conjunctivitis, peritonitis, pyelonephritis, pharyngitis, septic arthritis, phlegmon, epiglottitis, salpingitis, tympanitis, cold, flu, enteritis, dysentery and gastroenteritis.

The term “therapeutically effective amount” refers to a dosage level of about 0.5 mg to about 50 mg per kg body weight per day (typically from about 30 mg to about 3 g/patient/day) for the purpose of being used in treatment of the symptoms in humans.

The term “prophylactically effective amount” refers to a dosage level of about 0.1 mg to about 10 mg per kg body weight per day (typically from about 6 mg to about 0.6 g/patient/day) for the purpose of being used in prevention of the symptoms in humans. An administration duration should be at least 2 to 5 days and 1 to 6 weeks.

An amount of protein that may be combined with the carrier material to form a single dosage form may vary depending on a host to be treated and a particular mode of administration. For example, a formulation intended for oral administration to a human may contain 0.5 mg to 3 g of protein in combination with a suitable and convenient amount of a pharmaceutically acceptable carrier that may be included in an amount of about 1% to about 99% of the total composition. A unit dosage form generally contains about 0.5 mg to about 3 g of protein.

However, it may be appreciated that a specific effective amount for a particular patient may vary according to a variety of factors including an activity of a particular compound used, age, weight, general health, sex, diet, time of administration, route of administration, rate of excretion, drug combination, and severity of a certain disease to be prevented or treated. The pharmaceutical composition according to the present invention may be formulated into pill, sugar-coated tablet, capsule, liquid, gel, syrup, slurry, and suspension.

In a preferred embodiment, the pharmaceutical composition for oral administration may be prepared by mixing the active ingredient together with a solid excipient and may be prepared in a granule form to prepare a tablet form or a sugar-coated tablet form. As a suitable excipient, sugar forms such as lactose, sucrose, mannitol and sorbitol or starch from corn, wheat flour, rice, potato or other plants, cellulose such as methylcellulose, hydroxyclopropylmethyl-cellulose or sodium carboxymethylcellulose, carbohydrate such as gum including arabic gum or tragacanth gum, or protein filler such as gelatin or collagen, may be used. If necessary, a disintegrant or a solubilizer in each salt form such as cross-linked polyvinylpyrrolidone, agar and alginic acid or sodium alginic acid may be added.

In a preferred embodiment, for parenteral administration, the pharmaceutical composition of the present invention may be prepared as a water-soluble solution. Preferably, a physically suitable buffer solution such as Hank's solution, Ringer's solution or physically buffered brine may be used. A water-soluble injection suspension may contain a substrate capable of increasing a viscosity of the suspension, such as sodium carboxymethyl cellulose, sorbitol or dextran. In addition, a suspension of an active ingredient may be prepared as a suitable oily injection suspension. A suitable lipophilic solvent or carrier includes a fatty acid such as sesame oil or a synthetic fatty acid ester such as ethyl oleate, triglyceride or liposome. A polycationic amino polymer may also be used as a vehicle. Optionally, in the suspension, a suitable stabilizing agent or agent may be used to increase solubility of the compound and to prepare a high concentration of solution.

A protein in the pharmaceutical composition adsorbs to a glass container such as a vial, a syringe, or the like, is unstable, and easily deactivated by various physicochemical factors such as heat, pH, and humidity, etc. Therefore, a stabilizer, a pH adjuster, a buffer, a solubilizing agent, a surfactant, or the like, is added for formulation in a stable form. Examples of the stabilizer include amino acids such as glycine, alanine, etc., sugars such as dextran 40, mannose, etc., sugar alcohols such as sorbitol, mannitol, xylitol, etc., and further two or more thereof may be used in combination. An added amount of the stabilizer is preferably 0.01 to 100 times, more preferably 0.1 to 10 times, a weight of a protein. By adding the stabilizer, storage stability of a liquid preparation or a freeze-dried preparation may be improved. Examples of the buffer include a phosphate buffer, a citrate buffer, and the like. The buffer adjusts pH of the water-soluble solution and contributes to the stability and solubility of the protein after redissolution of the liquid preparation or the freeze-dried preparation. An added amount of the buffer is, for example, 1 to 10 mM relative to an amount of the liquid preparation or the freeze-dried preparation after the redissolution. As a surfactant, polysorbate 20, pulluronic F-68, polyethylene glycol, etc., may be used, and polysorbate 80 is particularly preferred, and further two or more thereof may be used in combination.

As described above, since a polymer protein is easily adsorbed to glass, resin, or the like, which is a material of a container, adsorption of the protein to the container after the redissolution of the liquid preparation or freeze-dried preparation may be prevented by adding the surfactant. An added amount of the surfactant is preferably 0.001 to 1.0% based on a weight of water after redissolving of the liquid formulation or the freeze-dried formulation. The formulation of the protein of the present invention may be prepared by adding the stabilizer, the buffer or the adsorption inhibitor as described above. In particular, when it is used as an injection for medical use or for animal medicine, an osmotic pressure ratio which is acceptable as an osmotic pressure ratio is preferably 1 to 2. The osmotic pressure ratio may be adjusted by increasing or decreasing sodium chloride during formation of medicine.

In another aspect, the present invention relates to a method for treating a herpes simplex virus infection characterized by treating a complex of interleukin-15 and interleukin-15 receptor alpha-Fc.

Hereinafter, the present invention is described in detail with reference to the following Examples. However, these following Examples are only for exemplifying the present invention, and it will be obvious to those skilled in the art that the scope of the present invention is not construed to be limited to these examples.

Hereinafter, effects of the complex of interleukin-15 and interleukin-15 receptor alpha-Fc in a mouse on HSV symptoms were confirmed through Examples using the mouse. However, it is obvious to those skilled in the art that the same or similar effects could be exhibited in HSV infections in mammals including human other than mouse even when using the complex of interleukin-15 and interleukin-15 receptor alpha-Fc of the corresponding mammals.

### Example 1: Production of HSV Symptomatic Model Mouse

To produce an HSV symptomatic model mouse, an ICR male mouse (Coatec, Orient) aged 4-5 weeks was infected with HSV type 1 (1×10⁶ pfu/mL, F strain (Korea National Institute of Health) cultured in Vero cells (ATCC). Virus inoculation was performed twice at an interval of 10 days. After second HSV inoculation, the mouse was observed from 4 to 32 weeks. The animal was treated according to a protocol approved by Animal Care committee at Aju Medical University.

After the HSV inoculation, the mouse exhibited various symptoms. The exhibited symptoms were mouth and genital ulcers, erythema, skin pustule, skin ulcer, arthritis, diarrhea, red eyes, eyesight deterioration, loss of balance, discoloration, facial edema, etc. Each symptom score was 1 point each and the total score of symptoms was used to determine the severity of symptom. Loss of the symptom or a reduction in lesion size of ≥20% was shown as being improved. As a control group, an HSV-inoculated but asymptomatic healthy mouse was used as a non-symptomatic control group (NS).

### Example 2: Confirmation of Expression of IL-15 and IL-15Rα in HSV Symptomatic Mouse

Peripheral blood mononuclear cells (PBMC) of non-symptomatic mouse (NS) and symptomatic mouse (S) after HSV inoculation were isolated, and mRNA expression of mouse IL-encoding gene and mouse IL-15Rα encoding gene in the PBMC cells were confirmed by RT-PCR.

Total RNA was isolated from the peripheral blood mononuclear cells (PBMC) of the mouse using TRIzol (Life Technologies, Hel-german, CT). Total RNA (1 μg) was used as a template for cDNA synthesis, and cDNA was synthesized using AccuPower RT Premix for RT-PCR (Bioneer, Daejeon, Korea).

Primer sequences used for RT-PCR were as follows:

Beta-actin:
forward,
(SEQ ID NO: 1)
5′-TGG AAT CCT GTG GCA TCC ATG AAA C-3′
reverse,
(SEQ ID NO: 2)
5′-TAA AAC GCA GCT CAG TAA CAG TCC G-3′
mIL-15,
forward,
(SEQ ID NO: 3)
5′-GAA TAC ATC CAT CTC GTG CTA CT-3
reverse,
(SEQ ID NO: 4)
5′-TTT GCA AAA ACT CTG TGA AGG-3′
mIL-15Rα
Forward,
(SEQ ID NO: 5)
5′-TTG CTG CTG CTG CTG TTG CTA CTG TTG CTC
CC-3′
Reverse,
(SEQ ID NO: 6)
5′-CA CCA ACC AAG AGG ACC GAT GTA GAG ATG
GC-3′

The amplified PCR product was visualized on a 1.5% agarose gel.

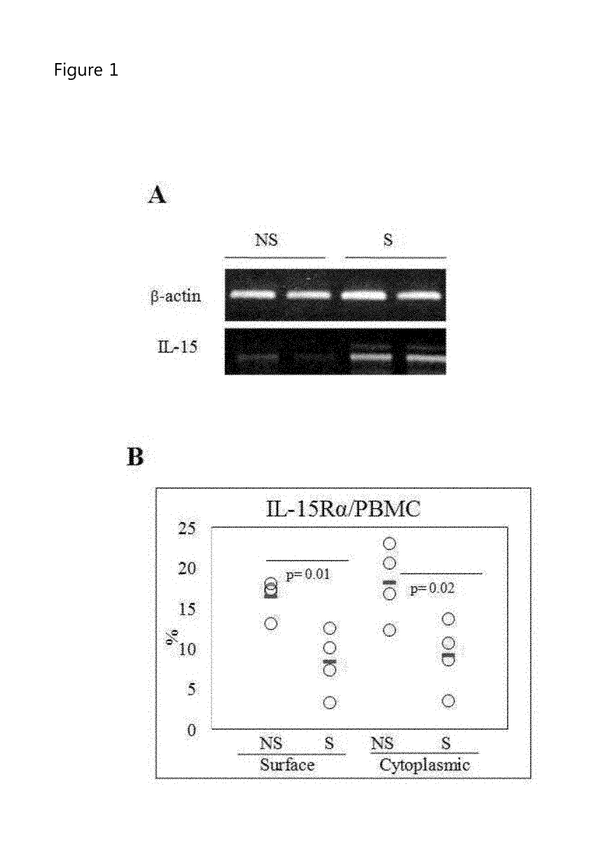

As a result, IL-15 mRNA expression was higher in the symptomatic mouse (S) than in the non-symptomatic mouse (NS), as shown in FIG. 1, in image A.

In addition, expression of IL-15Rα on cell surfaces and cytoplasm of cells derived from the non-symptomatic mouse (NS) and the symptomatic mouse (S) was analyzed by flow cytometry (FIG. 1, in graph B.

First, the peripheral blood mononuclear cells (PBMC), peritoneal macrophage, and lymph node (LN) cells of the mice were washed with PBS. Then, 1×10⁶ cells were treated with APC-labeled anti-IL-15Rα, FITC-labeled anti-CD4, Percp-Cy5.5-labeled anti-CD8, FITC-labeled anti-CD122, PE-labeled anti-CD132, PE-Cy7-labeled anti-CD11b, APC-labeled anti-CD27, PE-labeled anti-CD25, PE-labeled anti-NK1.1 (BD Pharmingen) for 30 minutes at 4° C. Foxp3 was labeled with PE-Cy7 by intracellular staining after perforation for 30 minutes at 4° C. (eBiosciences, San Diego, Calif., USA). The stained cells were analyzed by a FACS Aria III flow cytometer (Becton Dickinson, San Jose, Calif., USA) with ×10,000 gate cells.

As a result, it was confirmed that the frequency of IL-15Rα in the symptomatic mouse (S) was significantly lower than that in the non-symptomatic mouse (NS) in both cell surface expression and cytoplasmic expression in PBMC.

### Example 3: IL-15/IL-15Rα Vector Administration to Symptomatic Mouse

RAW 264.7 cells (ATCC) were transfected in vitro with pIL-15Rα or pIL-15/15Rα. The RAW 264.7 cells were cultured in DMEM media containing 10% FBS and 1% penicillin/streptomycin, plated in a 6-well plate at 1×10⁶ cells/well, and treated with 2 μg of vector mixed with a 3 μL FuGENE HD transfection reagent (Promega, Woods Hollow Road, Madison, Wis., USA). After incubation for 24 hours, expression was confirmed by RT-PCR.

The expression of IL-15Rα in transfected cells was confirmed by RT-PCR in the same manner as in Example 2.

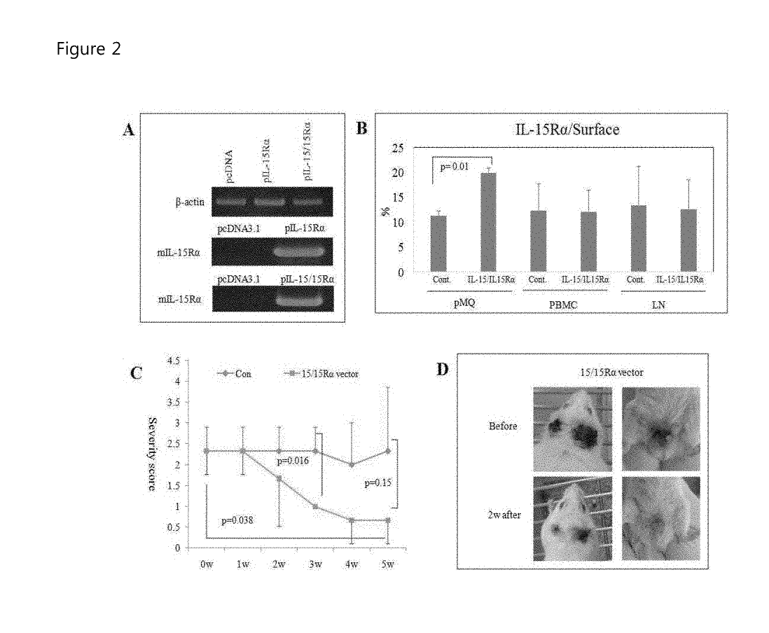

As a result, as shown in FIG. 2, in panel A, it was confirmed that IL-15Rα mRNA was expressed in the RAW 264.7 cells transfected with the IL-15Rα expression vector (pIL-15Rα, provided by Prof. Wu T C, The Johns Hopkins University School of Medicine) and the IL-15/IL-15Rα expression vector (pIL-15/15Rα, provided by Prof. Wu T C, The Johns Hopkins University School of Medicine).

The IL-15/IL-15Rα expression vector was intraperitoneally injected twice in BD mouse together with a transfection reagent, jetPEI (Polyplus Transfection, New York, USA), wherein the mouse received 50 μg of the first injection, and after 7 days, 100 μg of the second injection. At 14 days after the first injection, the treated BD mouse was subjected to FACS analysis by the method described in Example 2.

As a result, as shown in FIG. 2, graph B, the combined administration of pIL-15/15Rα to the healthy normal mouse was induced to up-regulate the frequency of IL-15Rα+ cells in the peritoneal macrophage of the mouse. In addition, pIL-15/15Rα injection significantly reduced disease severity scores after 3 weeks in the symptomatic mouse group compared to the control group (FIG. 2, in graph C). FIG. 2, in panel D shows an image evidencing a change in the symptom of the mouse after injection of pIL-15/15Rα.

### Example 4: IL-15/IL-15Rα-Fc Protein Complex Administration to Mouse

The rmIL-15/IL-15Rα-Fc protein complex was intravenously administered to the HSV-infected mouse group, and a change in immunological feature were confirmed by FACS.

A mixing ratio of IL-15 and IL-15Rα was set to IL-15:IL-15Rα-Fc=1:5.

The rmIL-15/IL-15Rα-Fc protein complex (IL-15, eBioscience cat no. 14-8153; IL-15Rα-Fc, R&D Systems cat no. 551-MR) in which 1.2 μg of recombinant IL-15 was mixed with 6 μg of IL-15Rα-Fc was intravenously injected for 2 days, consecutively, into HSV-infected mouse established in Example 1, and on day 3, the mouse was subjected to FACS analysis. For control drug, Colchicine (2 μg/mouse) was orally administered to the BD mouse for 5 days, consecutively. For control protein drug treatment, Infliximab (150 μg/mouse) was injected intravenously into the BD mouse once.

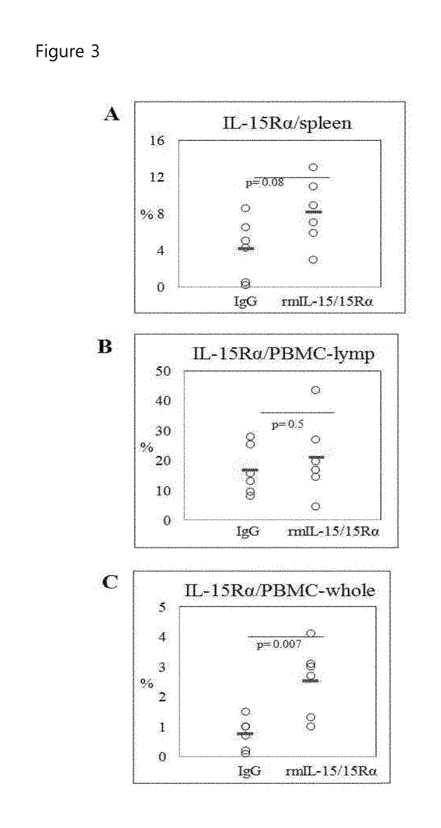

Intravenous treatment of the recombinant protein IL-15/IL-15Rα-Fc complex in the symptomatic mouse group improved the frequency of IL-15Rα+ cells in spleen and peripheral blood mononuclear cells (PBMC) (FIG. 3).

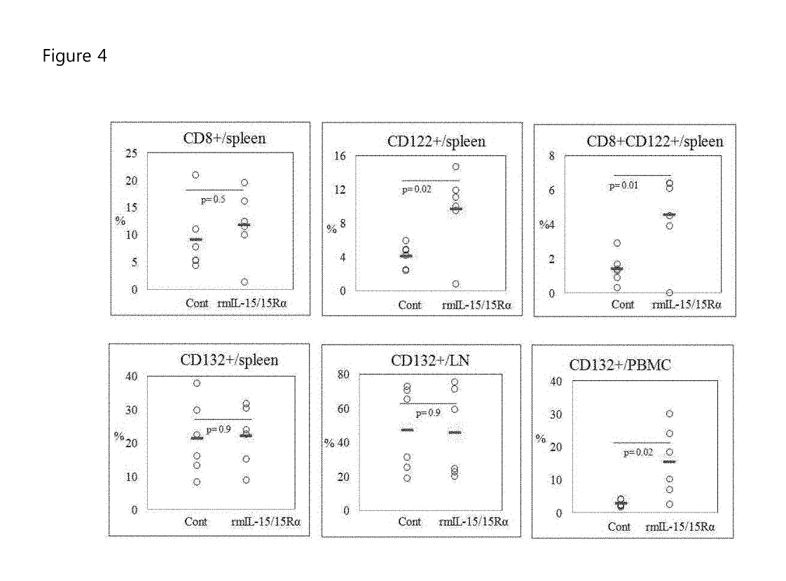

Treatment of the recombinant protein IL-15/IL-15Rα-Fc complex significantly increased the frequency of CD122+ cells (IL-15R beta) in the spleen of the symptomatic mouse (FIG. 4, in graph A). The frequency of CD132+ cells (IL-15R gamma) was increased in the PBMC of the mouse.

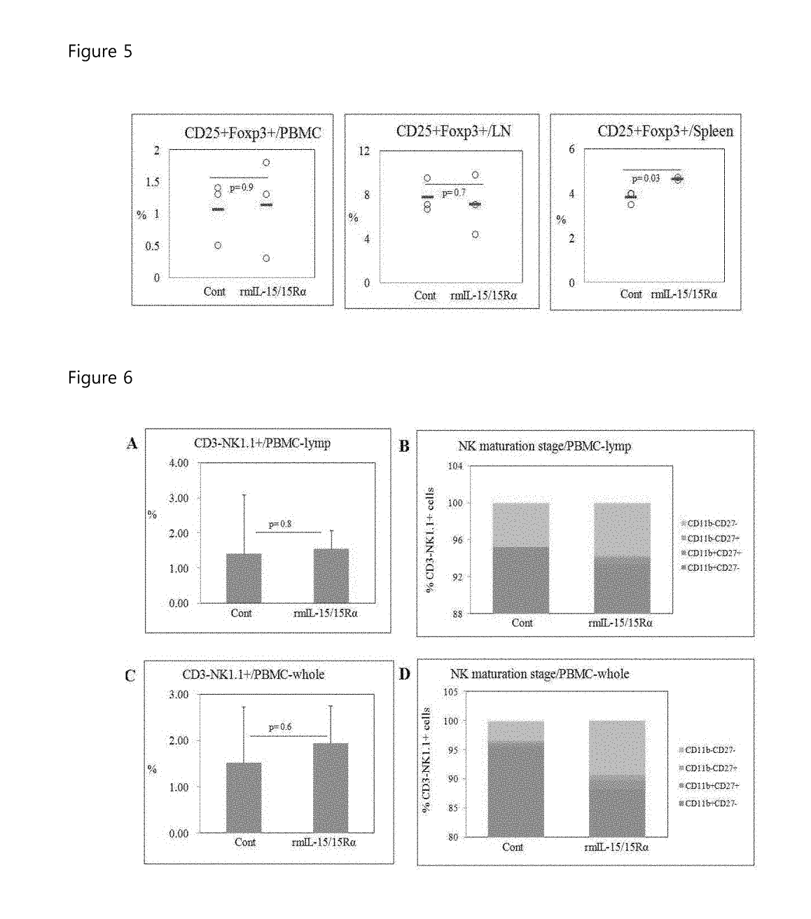

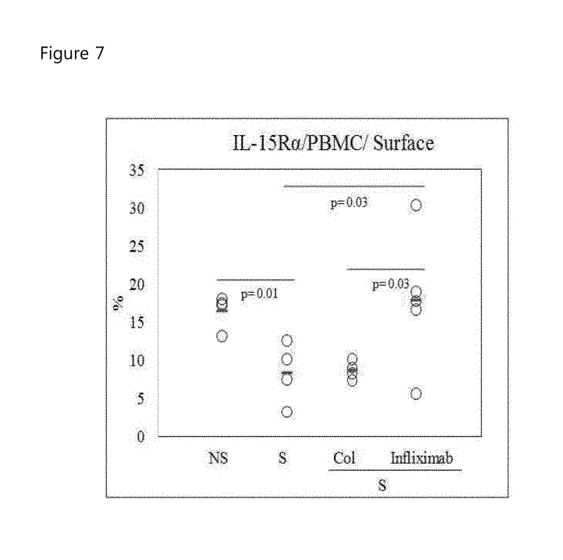

In addition, the frequency of CD25+/Foxp3+ cells was significantly increased in the spleen of the symptomatic mouse by treatment with the recombinant protein IL-15/IL-15Rα complex (FIG. 5).

Further, the treatment of the recombinant protein IL-15/IL-15Rα complex in the symptomatic mouse group was induced to up-regulate the frequency of CD11b−/CD27− cells, CD11b−/CD27+ cells, and CD11b+/CD27+ cells, but the frequency of CD11b+/CD27− cells was down-regulated in whole cells of PBMC and lymphocytes of PBMC (FIG. 6). The CD11b+/CD27− cell population is a population of terminally differentiated NK cells in four stages of NK cell differentiation.

Therefore, it may be considered that treatment of the IL-15/IL-15Rα-Fc complex inhibits maturation of the NK cells, which leads to the rejuvenation of NK cells.

Colchicine and infiliximab used in the present experiment are existing medicines that are prescribed for controlling inflammation, and the frequency of IL-15Rα+ cells in surface staining of PBMC was increased in the infliximab treated symptomatic mouse.

All data in the Examples above were expressed as mean standard deviation (SD), and statistical differences between the experimental groups were determined using Student's t test and Bonferroni correction. Statistical analysis was performed using MedCalc version 9.3.0.0 (MedCalc, Ostend, Belgium) and statistical significance was considered when the p-value was less than 0.05.

According to the present invention, the herpes simplex virus infections such as herpes labialis, angular herpes, angular cheilitis, genital herpes simplex, ocular herpes, brain herpes infectious encephalitis, Mollaret's meningitis, and neonatal herpes, etc., may be fundamentally treated by intravenous administration as well as topical administration.

The present invention has been described in detail based on particular features thereof, and it is obvious to those skilled in the art that these specific technologies are merely preferable embodiments and thus the scope of the present invention is not limited to the embodiments. Therefore, the substantial scope of the present invention is defined by the accompanying claims and equivalent thereof.

1. A method for treating or preventing infectious disease caused by herpes simplex virus, the method comprising: administering a composition containing interleukin-15 and interleukin-15 receptor alpha-Fc to a subject in need of treatment or prevention of said disease.

2. The method of claim 1, wherein the concentration of interleukin-15 is 0.01-10 mg/mL in the composition.

3. The method of claim 1, wherein the concentration of interleukin-15 receptor alpha-Fc is 0.01-60 mg/mL in the composition.

4. The method of claim 1, wherein the content ratio of interleukin-15 and interleukin-15 receptor alpha-Fc is 0.1-5:5-50.

5. The method of claim 1, wherein the infectious disease caused by herpes simplex virus is selected from the group consisting of herpes labialis, angular herpes, genital herpes simplex, ocular herpes, brain herpes infectious encephalitis, Mollaret's meningitis, and neonatal herpes.

6. The method of claim 1, wherein the composition is administered intravenously, subcutaneously, or intramuscularly.

7. The method of claim 1, wherein the composition is topically administered.

8. The method of claim 1, wherein the interleukin-15 and the interleukin-15 receptor alpha-Fc are contained in said composition in the form of purified protein.

9. The method of claim 1, wherein the interleukin-15 and the interleukin-15 receptor alpha-Fc are contained in said composition in the form of recombinant vector(s) of the interleukin-15 and/or the interleukin-15 receptor alpha-Fc.


SEQUENCE LISTING (6 sequences)

SEQ ID NO: 1 - DNA, 25 bp, Artificial Sequence
  primer
tggaatcctgtggcatccatgaaac

SEQ ID NO: 2 - DNA, 25 bp, Artificial Sequence
  primer
taaaacgcagctcagtaacagtccg

SEQ ID NO: 3 - DNA, 23 bp, Artificial Sequence
  primer
gaatacatccatctcgtgctact

SEQ ID NO: 4 - DNA, 21 bp, Artificial Sequence
  primer
tttgcaaaaactctgtgaagg

SEQ ID NO: 5 - DNA, 32 bp, Artificial Sequence
  primer
ttgctgctgctgctgttgctactgttgctccc

SEQ ID NO: 6 - DNA, 31 bp, Artificial Sequence
  primer
caccaaccaagaggaccgatgtagagatggc